(19) United States
(12) Patent Application Publication
(10) Pub. No.: US 2011/0184107 A1
(43) Pub. Date: Jul. 28, 2011

(54) FLAME RETARDANT HALOGENATED PHENYL ETHERS

(75) Inventor: Larry D. Timberlake, West Lafayette, IN (US)

(73) Assignee: CHEMTURA CORPORATION, Middlebury, CT (US)

(21) Appl. No.: 12/692,887

(22) Filed: Jan. 25, 2010

(51) Int. Cl. C08K 5/06; C07C 43/205

(52) U.S. Cl. 524/371; 568/637

(57) ABSTRACT

A halogenated non-polymeric phenyl ether is described having the general formula (I):

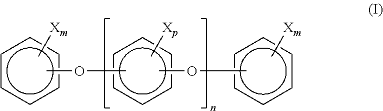

wherein each X is independently Cl or Br, n is an integer of 1 or 2, each m is independently an integer of 1 to 5 and each p is independently an integer of 1 to 4, provided that, when X is Cl, the total amount halogen in the ether is from about 50 to about 65 wt % and when, X is Br, the total amount halogen in the ether is from at least 70 wt % to about 79 wt %.

### FIELD

This invention relates to flame retardant halogenated phenyl ethers.

### BACKGROUND

Decabromodiphenyl oxide (deca) and decabromodiphenylethane (deca-DPE) are commercially available materials widely used to flame retard various polymer resin systems. The structure of these materials is as follows:

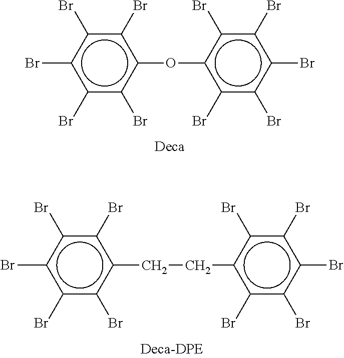

One of the advantages of using deca and deca-DPE in polymer resins that are difficult to flame retard, such as high-impact polystyrene (HIPS) and polyolefins, is that the materials have a very high (82-83%) bromine content. This allows a lower load level in the overall formulation, which in turn serves to minimize any negative effects of the flame retardant on the mechanical properties of the polymer.

Despite the commercial success of deca, there remains significant interest in developing alternative halogenated flame retardant materials that are equally or more efficient, not only because of economic pressures but also because they may allow lower flame retardant loadings, which in turn may impart improved performance properties. Improved properties, such as non-blooming formulations, or better mechanical properties can potentially be met by producing polymeric or oligomeric flame retardant compounds. These types of material tend to become entangled in the base resin polymer matrix, depending on the compatibility between the resin and the flame retardant, and hence should show fewer tendencies to bloom.

There are a number of commercially available flame retardant materials that can be considered oligomers or polymers of halogenated monomers. Examples of such halogenated monomers include tetrabromobisphenol A (TBBPA) and dibromostyrene (DBS), which have the following structures:

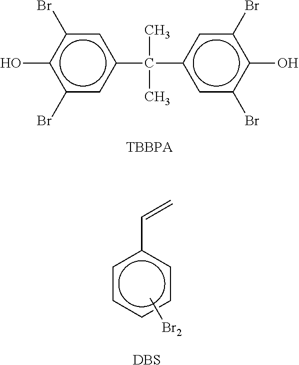

Commercially, TBBPA and DBS are typically not used in their monomeric form, but are converted into oligomeric or polymeric species. One class of oligomers is the brominated carbonate oligomers based on TBBPA. These are commercially available from Chemtura Corporation (examples include Great Lakes BC-52™, Great Lakes BC-52HP™, and Great Lakes BC-58™) and by Teijin Chemical (FireGuard 7500 and FireGuard 8500). These products are used primarily as flame retardants for polycarbonate and polyesters.

Brominated epoxy oligomers, based on condensation of TBBPA and epichlorohydrin, are commercially available and sold by Dainippon Ink and Chemicals under the Epiclon® series, and also by ICL Industrial Products (examples are F-2016 and F-2100) and other suppliers. The brominated epoxy oligomers find use as flame retardants for various thermoplastics both alone and in blends with other flame retardants.

Another class of brominated polymeric flame retardants based on TBBPA is exemplified by Teijin FG-3000, a copolymer of TBBPA and 1,2-dibromoethane. This aralkyl ether finds use in ABS and other styrenic polymers. Alternative end-groups, such as aryl or methoxy, on this polymer are also known as exemplified by materials described in U.S. Pat. No. 4,258,175 and U.S. Pat. No. 5,530,044. The non-reactive end-groups are claimed to improve the thermal stability of the flame retardant.

TBBPA is also converted into many other different types of epoxy resin copolymer oligomers by chain-extension reactions with other difunctional epoxy resin compounds, for example, by reaction with the diglycidylether of bisphenol A. Typical examples of these types of epoxy resin products are D.E.R.™ 539 by the Dow Chemical Company, or Epon™ 828 by Hexion Corporation. These products are used mainly in the manufacture of printed circuit boards.

DBS is made for captive use by Chemtura Corporation and is sold as several different polymeric species (Great Lakes PDBS-80™, Great Lakes PBS-64HW™, and Firemaster CP44—HF™) to make poly(bromostyrene) type flame retardants. These materials represent homopolymers or copolymers. Additionally, similar brominated polystyrene type flame retardants are commercially available from Albemarle Chemical Corporation (Saytex® HP-3010, Saytex® HP-7010, and PyroChek 68PB). All these polymeric products are used to flame retard thermoplastics such as polyamides and polyesters.

Unfortunately, one of the key drawbacks of the existing halogenated polymer materials is their relatively low halogen content, which makes them less efficient as flame retardants and consequently typically has a negative effect on the desirable physical properties of the flame retardant formulations containing them, such as impact strength. For example, whereas deca and deca-DPE contain 82-83% bromine, oligomers or polymers based on the brominated monomers mentioned above generally have a bromine content in the range of 52%-68%, depending on the material. This therefore typically requires a flame retardant loading level in a polymer formulation significantly higher than that required for deca, often resulting in inferior mechanical properties for the formulation.

In our U.S. Patent Application Publication No. 2008/0269416, we have proposed a new class of flame retardant materials that to not detract from the mechanical properties of the target resin and that are based on halogenated aryl ether oligomers comprising the following repeating monomeric units:

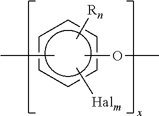

wherein R is hydrogen or alkyl, especially C₁ to C₄ alkyl, Hal is halogen, normally bromine, m is at least 1, n is 0 to 3 and x is at least 2, such as 3 to 100,000. These materials can be halogenated to a higher level than other currently available oligomeric flame retardants and provide superior mechanical properties when combined with resins such as HIPS and polyolefins as well as engineering thermoplastics such as polyamides and polyesters. It is also found that these aryl ether oligomers, even at lower levels of halogenation, give formulations with acceptable mechanical properties.

The materials disclosed in the '416 publication are polymeric in the sense that they have a molecular weight distribution resulting from the varying degrees of polymerization of the monomer units. In addition, it is known that certain discrete halogenated phenyl ether compounds, which have multiple phenyloxy linkages but which are not polymeric in the sense that they do not have a molecular weight distribution, have utility as flame retardants. For example, Japanese Unexamined Patent Application Publication 2-129,137 discloses flame retardant polymer compositions in which the polymer is compounded with a halogenated bis(4-phenoxyphenyl)ether shown by general formula [I]:

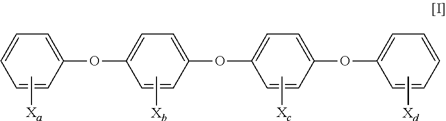

in which X is a halogen atom, a and d are numbers in the range of 1-5, and b and c are numbers in the range of 1-4. Materials containing from 64 to 81 wt % Br are exemplified in the application.

In addition, U.S. Pat. No. 3,760,003 discloses halogenated polyphenyl ethers having the general formula:

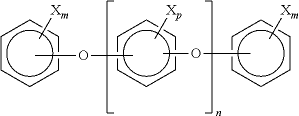

wherein each X is independently Cl or Br, each m is independently an integer of 0 to 5, each p is independently an integer of 0 to 4, n is an integer of 2 to 4, and 50% or more by weight of the compound is halogen. Perbrominated materials containing at least 60 wt % Br are said to be preferred.

Further the fully brominated polyphenyl ether (81.8 wt % Br), tetradecabromodiphenoxybenzene:

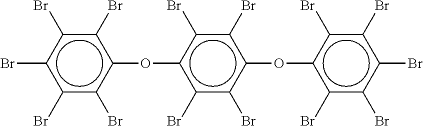

is sold by Albemarle Chemical Corporation under the trade name SAYTEX 120 for use as a flame retardant in high performance polyamide and linear polyester engineering resins and alloys, as well as in polyolefin and styrenic resins.

According to the present invention, it has now been found that certain non-polymeric phenyl ethers, when halogenated to a controlled level slightly below full halogenation, exhibit a unique combination of flame retardant efficiency and superior mechanical properties when combined with a wide variety of resin compositions.

### SUMMARY

In one aspect, the invention resides in a halogenated non-polymeric phenyl ether having the general formula (I):

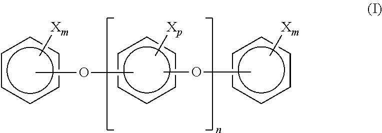

wherein each X is independently Cl or Br, n is an integer of 1 or 2, each m is independently an integer of 1 to 5 and each p is independently an integer of 1 to 4, provided that, when X is Cl, the total amount halogen in the ether is from about 50 to about 65 wt % and when, X is Br, the total amount halogen in the ether is from at least 70 wt % to about 79 wt %.

In one embodiment, at least one non-terminal phenyl group is connected to two phenoxy groups in the 1,4-positions. Alternatively, at least one non-terminal phenyl group can be connected to two phenoxy groups in the 1,3-positions or the 1,2-positions.

In a further aspect, the invention resides in a flame retardant polymer composition comprising (a) a flammable macromolecular material and (b) a flame retardant amount of a halogenated non-polymeric phenyl ether having the general formula (I):

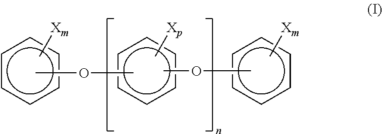

wherein each X is independently Cl or Br, n is an integer of 1 or 2, each m is independently an integer of 1 to 5 and each p is independently an integer of 1 to 4, provided that when X is Cl the total amount halogen in the ether is from about 50 to about 65 wt % and when X is Br the total amount halogen in the ether is from at least 70 wt % to about 79 wt %.

### DETAILED DESCRIPTION

Described herein is a partially halogenated non-polymeric phenyl ether and its use as a flame retardant for flammable macromolecular polymers. Suitable macromolecular polymers include thermoplastic polymers, such as polystyrene, poly (acrylonitrile butadiene styrene), polycarbonates, polyolefins, polyesters and polyamides, and thermosetting polymers, such as epoxy resins, unsaturated polyesters, polyurethanes and rubbers.

The term “non-polymeric phenyl ether” is used herein to mean a compound which has a fixed number of aryloxy linkages and hence a discrete molecular weight. This is in contrast to an aryl ether polymer or oligomer which has a molecular weight distribution resulting from the varying degrees of polymerization of its aryl ether monomer units.

The partially halogenated non-polymeric phenyl ether employed in the present blend has the general formula (I):

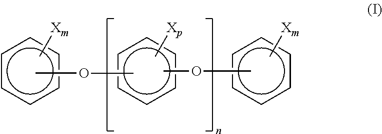

wherein each X is independently Cl or Br, n is an integer of 1 or 2, each m is independently an integer of 1 to 5 and each p is independently an integer of 1 to 4, provided that when X is Cl the total amount halogen in the ether is from about 50 to about 65 wt %, especially from about 60 wt % to about 64 wt %, and when X is Br the total amount halogen in the ether is from at least 70 wt % to about 79 wt %, especially from about 71 wt % to about 78 wt %, such as about 71 wt % to about 76 wt %.

In one embodiment, n is 1 and the partially halogenated non-polymeric phenyl ether has the formula (II):

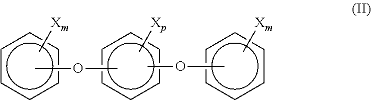

In another embodiment, n is 2 and the partially halogenated non-polymeric phenyl ether has the formula (III):

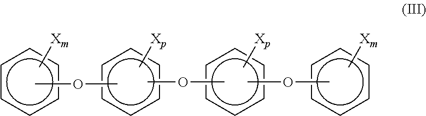

In each of the above embodiments, the phenoxy groups attached to the non-terminal phenyl groups may be totally or partially in the 1,4 (para)-position, the 1,3 (meta)-position or the 1,2 (ortho) position. For example, for a 3-ring phenyl ether of Formula (II), 3 configurations, para (3p), meta (3m) and ortho (3o), are possible for the phenoxy groups attached to the single non-terminal phenyl group:

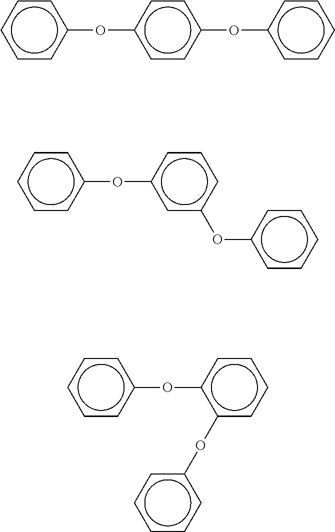

In the case of a 4-ring phenyl ether of formula (III), 6 configurations, 4 pp, 4 pm, 4 mm, 4po, 4mo and 400, are possible. Considering, for simplicity only the para and meta configurations, these are as follows:

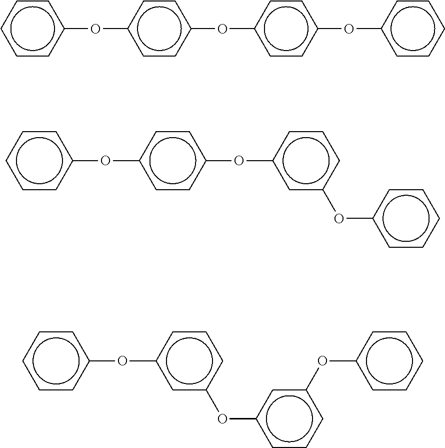

The level of bromination theoretically available for some of the 3-ring or 4-ring aryl ethers of formulas (II) and (III) is summarized as follows:

| | No. of Bromines | |
|---|---|---|
| Material | Present | % OBr |
| 3-Ring | 7 | 68.7 |
| | 8 | 74.5 |
| | 9 | 74.0 |
| | 10 | 76.0 |
| | 11 | 77.8 |
| | 12 | 79.3 |
| | 13 | 80.7 |
| | 14 (max. possible) | 81.8 |
| 4-Ring | 9 | 67.6 |
| | 10 | 69.9 |
| | 11 | 71.9 |
| | 12 | 73.7 |
| | 13 | 75.3 |
| | 14 | 76.7 |
| | 15 | 77.9 |
| | 16 | 79.0 |
| | 17 | 80.1 |
| | 18 (max. possible) | 81.1 |

Each of the partially halogenated non-polymeric phenyl ether described above is produced by halogenation, normally bromination, of its associated phenyl ether precursor, which in turn can be made from the appropriate aryl halide and aryl hydroxyl compounds by the Ullmann aryl ether synthesis. Details of the Ullmann aryl ether synthesis can be found in the literature. Some review articles on this subject include Ley, S. V. and Thomas, A. W. Angew. Chem. Int. Ed. 2003, 42, 5400-5449; Sawyer, J. S. Tetrahedron, 2000, 56, 5045-5065; Lindley, James Tetrahedron, 1984, 40(9), 1433-1456; and Frlan, R. and Kikelj, D. Synthesis, 2006, 14, 2271-2285.

Bromination of the resultant phenyl ether precursor is readily achieved by the reaction of the phenyl ether with bromine in the presence of a Lewis acid catalyst, such as aluminum chloride. Depending on the amount of bromine desired to be introduced into the phenyl ether, the weight ratio of bromine to oligomer employed in the bromination reaction is typically between about 3.5:1 and about 9.0:1, such as between about 4.5:1 and about 7.0:1. The degree of bromination is typically controlled by the bromine stoichiometry of the reaction. Alternatively, in cases where excess bromine is used, the degree of bromination is controlled by either reaction time and/or by monitoring the amount of by-product HBr that is produced. In that case, the reaction could be stopped when the target bromination level is reached by adding a small amount of water to kill the catalyst.

Alternatively, bromine chloride may be used as the brominating agent to generate the desired product in similar fashion. In this case, a small amount of organically-bound chlorine would also be present, but would not detract from the properties of the final flame retardant.

Because of its high thermal stability and its relatively high halogen content, the partially halogenated phenyl ether described herein can be used as a flame retardant for many different polymer resin systems. Surprisingly, the resultant flame retarded polymer systems are frequently found to exhibit the superior mechanical properties, such as impact strength, as compared with the same systems flame retarded with the fully halogenated ether counterpart. Generally, the halogenated phenyl ether is employed as a flame retardant with thermoplastic polymers, such as polystyrene, high-impact polystyrene (HIPS), poly (acrylonitrile butadiene styrene) (ABS), polycarbonates (PC), PC-ABS blends, polyolefins, polyesters and/or polyamides. With such polymers, the level of the partially halogenated phenyl ether in the polymer formulation required to give a V-0 classification when subjected to the flammability test protocol from Underwriters Laboratories is generally within the following ranges:

| Polymer | Useful | Preferred |
|---|---|---|
| Polystyrene | 5 to 25 wt % | 10 to 20 wt % |
| Polypropylene | 20 to 50 wt % | 25 to 40 wt % |
| Polyethylene | 5 to 35 wt % | 20 to 30 wt % |
| Polyamide | 5 to 25 wt % | 10 to 20 wt % |
| Polyester | 5 to 25 wt % | 10 to 20 wt %. |

The present halogenated phenyl ether can also be used with thermosetting polymers, such as epoxy resins, unsaturated polyesters, polyurethanes and/or rubbers. Where the base polymer is a thermosetting polymer, a suitable flammability-reducing amount of the halogenated phenyl ether is between about 5 wt % and about 35 wt %, such as between about 10 wt % and about 25 wt %.

Typical applications for polymer formulations containing the present halogenated phenyl ether as a flame retardant include automotive molded components, adhesives and sealants, fabric back coatings, electrical wire and cable jacketing, and electrical and electronic housings, components and connectors. In the area of building and construction, typical uses for the present flame retardant include self extinguishing polyfilms, wire jacketing for wire and cable, backcoating in carpeting and fabric including wall treatments, wood and other natural fiber-filled structural components, roofing materials including roofing membranes, roofing composite materials, and adhesives used to in construction of composite materials. In general consumer products, the present flame retardant can be used in formulation of appliance parts, housings and components for both attended and unattended appliances where flammability requirements demand.

The invention will now be more particularly described with reference to the following non-limiting Examples.

### Example 1

### Synthesis of Brominated 3p Phenyl Ether

1,4-diphenoxybenzene is prepared by the Ullmann ether synthesis as follows. 4-Phenoxyphenol (186.2 g, 1.0 mol) is dissolved in 1600 g of DMF with 300 mL toluene under nitrogen. A 50% KOH solution (112.0 g, 1.0 mol) is added followed by azeotropic removal of the water and stripping of the toluene. Bromobenzene (157.0 g, 1.0 mol) and cupric oxide (3.2 g, 0.04 mol) are then added and the reaction solution held at reflux (153° C.) for 24 hr. The DMF is then removed by stripping and the residue worked up to give 1,4-diphenoxybenzene.

Bromine (640.6 g) is added to a solution of 107.8 g of 1,4-diphenoxybenzene in 500 mL of dichloromethane containing 9.6 g of AlCl₃ catalyst. The reaction temperature is kept at 30° C. and the HBr off-gas is captured in a water trap. After the HBr evolution subsides, the material is worked up to give the product as an off-white solid. The material is analyzed to contain 72.6% bromine.

### Example 2

### Synthesis of Brominated 3p Phenyl Ether

The process of example 1 was repeated but with bromination being conducted by adding 334.8 g of bromine to a solution containing 50 g of 1,4-diphenoxybenzene with 5 g of AlCl₃ in 333 mL of chloroform. The material is analyzed to contain 74.2% bromine.

### Example 3

### Synthesis of Brominated 3m Phenyl Ether

The process of Example 1 is repeated but with the 4-phenoxyphenol being replaced by 3-phenoxyphenol.

### Examples 4 to 13

### Synthesis of Various Brominated Phenyl Ether Compounds

A similar procedure to that described in Example 1 is employed using the appropriate starting material substrate to generate the desired brominated aryl ether compounds, as shown in Table 1. The amount of bromine used was adjusted as needed.

### Example 14

### Compounding of Brominated Phenyl Ethers in HIPS Resin

The brominated phenyl ethers prepared in Examples 1 to 13 were compounded separately with HIPS (high impact polystyrene) resin formulations containing antimony oxide (ATO) synergist using a twin-screw extruder with barrel temperatures of 200 to 220° C. For comparison, a similar formulation was prepared using decabromodiphenyl oxide (“deca”) as the flame retardant. The resultant formulations were injection-molded into test bars and evaluated as shown in Table 1. The Izod Notched Impact Strength (N-Impact in Table 1) values were measured according to ASTM D-256.

TABLE 1

| | | HIPS Results | | | | |
|---|---|---|---|---|---|---|
| | | | | | MFI, | |
| Brominated | | | | Vicat, | g/10 min | |
| Aryl Ether | | | N-Impact | ° C. | (200° C., | |
| Example | Structure | % Br | ft-lb/in | (10 N) | 5 Kg) | UL-94 |
| Deca | 2 | 83 | 2.1 | 96.3 | 11.6 | V-0 |
| 1 | 3p | 72.6 | 2.8 | 94.0 | 19.6 | V-0 |
| 2 | 3p | 74.2 | 3.0 | — | — | V-0 |
| 3 | 3m | 72.4 | 3.3 | — | — | V-0 |
| 4 | 3p | 79.6 | 1.9 | 97.4 | 8.5 | V-0 |
| 5 | 4pp | 70.8 | 2.3 | 97.0 | 17.3 | V-0 |
| 6 | 4mp | 74.7 | 2.9 | — | — | V-0 |
| 7 | 4mp | 70.7 | 3.0 | — | — | V-0 |
| 8 | 4pp | 75.1 | 2.8 | — | — | V-0 |
| 9 | 4pp | 79.5 | 1.35 | 97.5 | 8.8 | V-0 |
| 10 | 5ppp | 75.3 | 0.8 | 98.2 | 8.2 | V-0 |
| 11 | 5ppp | 78.8 | 1.30 | 95.9 | 9.9 | V-0 |
| 12 | 5pmp | 75.3 | 1.2 | — | — | V-0 |
| 13 | 5mmm | 73.1 | 2.6 | — | — | V-0 |

As shown in Table 1, in both cases for 3 and 4-ring aryl ethers, the impact strength properties were inferior for the pure materials at the higher bromine levels. However, if slightly underbrominated, the impact strength improved. There is also an observed increase in the melt flow properties. Based on this data, the number of bromines substituted on the 3-ring aryl ether should be less than around 12 per molecule (˜79.3%) to achieve satisfactory impact strength properties. Likewise, the number of bromines substituted on the 4-ring aryl ether should be less than around 16 per molecule (˜79.0%) to achieve satisfactory impact strength.

Surprisingly, the same improvement in impact strength is not observed with the partially and more fully brominated 5-ring and higher aryl ethers. The influence of meta vs para substitution in this case seems to be a more dominant effect.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What we claim is:

1. A halogenated non-polymeric phenyl ether having the general formula (I): 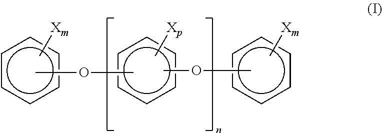
wherein each X is independently Cl or Br, n is an integer of 1 or 2, each m is independently an integer of 1 to 5 and each p is independently an integer of 1 to 4, provided that, when X is Cl, the total amount halogen in the ether is from about 50 to about 65 wt % and when, X is Br, the total amount halogen in the ether is from at least 70 wt % to about 79 wt %.

2. The ether of claim 1, wherein n is 1.

3. The ether of claim 1, wherein n is 2.

4. The ether of claim 1, wherein each X is bromine.

5. The ether of claim 1, wherein at least one non-terminal phenyl group is connected to two phenoxy groups in the 1,4-positions.

6. The ether of claim 1, wherein at least one non-terminal phenyl group is connected to two phenoxy groups in the 1,3-positions.

7. The ether of claim 1, wherein at least one non-terminal phenyl group is connected to two phenoxy groups in the 1,2-positions.

8. A flame retardant polymer composition comprising (a) a flammable macromolecular material and (b) a flame retardant amount of a halogenated non-polymeric phenyl ether having the general formula (I): 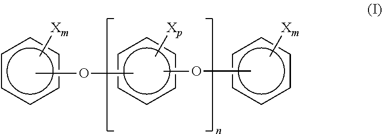
wherein each X is independently Cl or Br, n is an integer of 1 or 2, each m is independently an integer of 1 to 5 and each p is independently an integer of 1 to 4, provided that when X is Cl the total amount halogen in the ether is from about 50 to about 65 wt % and when X is Br the total amount halogen in the ether is from at least 70 wt % to about 79 wt %.

9. The composition of claim 8, wherein n is 1.

10. The composition of claim 8, wherein n is 2.

11. The composition of claim 8, wherein each X is bromine.

12. The composition of claim 8, wherein at least one non-terminal phenyl group is connected to two phenoxy groups in the 1,4-positions.

13. The composition of claim 8, wherein at least one non-terminal phenyl group is connected to two phenoxy groups in the 1,3-positions.

14. The composition of claim 8, wherein at least one non-terminal phenyl group is connected to two phenoxy groups in the 1,2-positions.

15. The composition of claim 8, wherein the flammable macromolecular material (a) is a thermoplastic polymer or a thermosetting polymer.

16. The composition of claim 8, wherein the flammable macromolecular material (a) is polystyrene and the amount of flame retardant is between about 5 and about 25 wt % of the composition.

17. The composition of claim 8, wherein the flammable macromolecular material (a) is polypropylene and the amount of flame retardant is between about 20 and about 50 wt % of the composition.

18. The composition of claim 8, wherein the flammable macromolecular material (a) is polyethylene and the amount of flame retardant is between about 5 and about 35 wt % of the composition.

19. The composition of claim 8, wherein the flammable macromolecular material (a) is a polyamide or polyester and the amount of flame retardant is between about 5 and about 25 wt % of the composition.
